(19) United States
(12) Patent Application Publication
(10) Pub. No.: US 2022/0370218 A1
(43) Pub. Date: Nov. 24, 2022

(54) Brain Aneurysm Manager

(71) Applicant: Rassan Mohammad Tarabein, Daphne, AL (US)

(72) Inventor: Rassan Mohammad Tarabein, Daphne, AL (US)

(21) Appl. No.: 17/843,826

(22) Filed: Jun. 17, 2022

(51) Int. Cl. A61F 2/91; A61F 2/88

(52) U.S. Cl. CPC A61F 2/91; A61F 2/88; A61F 2250/0067

(57) ABSTRACT

Brain Aneurysm Repair kit is a device developed to treat SAH (Sub-arachnoid Hemorrhage). It seals arterial damaged wall from ruptured brain aneurysms, stops the bleeding into the Sub-arachnoid space, and restores flow in the fastest way possible. Once the Cath is introduced, a dye is injected to identify the ruptured aneurysm site, a silicone-cover coated stent with a sticky adhesive on its out-surface is deployed, then the stent is inflated with a balloon against the ruptured arterial wall to stop the bleed, the sticky surface of the stent's silicone is secured against the arterial wall to close off the broken arterial wall and restore blood flow.

Brain Aneurysm Manager is a device designed to manage and treat subarachnoid hemorrhage because of a ruptured aneurysm. Nonetheless, the device may be used to prevent the rupture should an aneurysm be discovered coincidentally.

### BACKGROUND

Ruptured brain aneurysms are fatal in about 50% of cases. Of those who survive, about 66% suffer some permanent neurological deficit. Approximately 15% of people with a ruptured aneurysm die before reaching the hospital. Most deaths are due to rapid and massive brain injury from the initial bleeding. Thus, bleeding prevention and restoring blood flow in the ruptured aneurysm artery ought to be the top priority in management.

### DISCLOSURE OF THE BRAIN ANEURYSM MANAGER

this is the first Device to specifically address the treatment of subarachnoid hemorrhage via the repair of the arterial wall damage caused by the ruptured aneurysm and restoration of the blood flow to avert ischemic stroke distal to the site of the aneurysm.

In essence, stopping the bleed, sealing the damage, and restoring blood flow all could be achieved in the fastest way possible using the Brain Aneurysm Manager.

The Device entails using a catheter (FIG. 1) that can be introduced through the femoral or the radial arteries. Once positioned at the ruptured aneurysm site under radiographic guidance, a silicone cover-coated stent is deployed via balloon (FIG. 2) to seal off the damaged arterial wall and stop the bleeding into the subarachnoid space.

Once the balloon is deflated and the Cath is withdrawn (FIG. 3), the blood flow to the brain tissue distal to the ruptured aneurysm is restored.

### DESCRIPTION OF THE DRAWINGS

FIG. 1: The Brain Aneurysm Manager device is introduced via a guide wire and “snaked” to the aneurysm site.

FIG. 2: The stent is deployed by the balloon till the silicone sleeve stretches to seal the artery's walls, where the ruptured aneurysm is, to stop the bleed.

FIG. 3: The Balloon is deflated, and the Device is withdrawn, allowing the restoration of blood flow to the ischemic distal tissue to perfuse and revive.

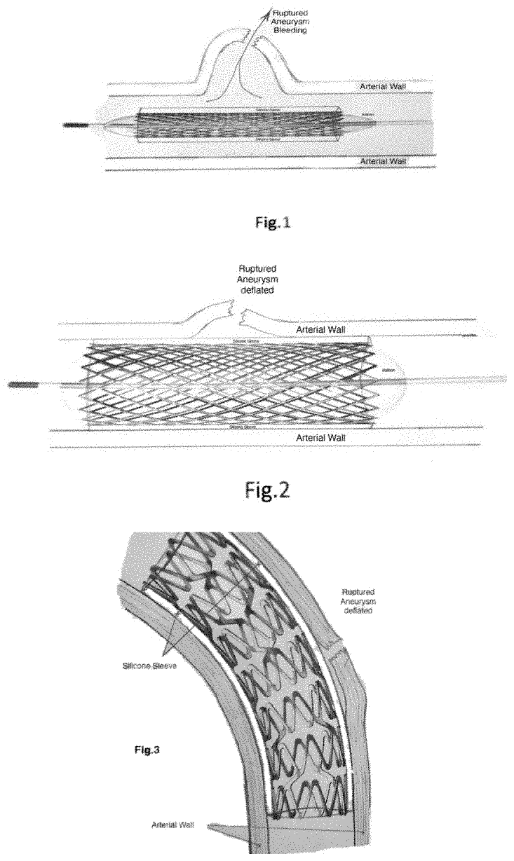

1. Brain Aneurysm Manager (as described in the application) is the first and only device to treat actively bleeding brain aneurysms.

2. Brain Aneurysm Manager can treat any aneurysm, whether ruptured or not.

3. Brain Aneurysm Manager is produced in different sizes and lengths to fit the artery size as well as the size of the aneurysm.
